(19) United States
(12) Patent Application Publication
(10) Pub. No.: US 2025/0013920 A1
(43) Pub. Date: Jan. 9, 2025

(54) SYSTEM AND METHOD FOR GENERATING A RECIPE FOR A THERMOPLASTIC COMPOUND

(71) Applicant: DSM IP Assets B.V., Heerlen (NL)

(72) Inventors: Ellen VAN HEMELRIJCK, ECHT (NL); Angelika SCHMIDT, ECHT (NL); Willem GODLIEB, ECHT (NL); Erwin Johannes Elisabeth HOUBEN, ECHT (NL); Jelle Fokke DE JONG, ECHT (NL); Arnold Theodoor Marie WILBERS, ECHT (NL)

(21) Appl. No.: 18/709,689

(22) PCT Filed: Oct. 3, 2022
(86) PCT No.: PCT/EP2022/077457

§ 371 Date: May 13, 2024

(30) Foreign Application Priority Data
Nov. 16, 2021 (EP) 21208386.9

(51) Int. Cl. G06N 20/00

(52) U.S. Cl. CPC G06N 20/00

(57) ABSTRACT

A processor system and method (100) are provided for generating a recipe for a thermoplastic compound, wherein the recipe defines a set of ingredients and a relative contribution of the ingredients for manufacturing the thermoplastic compound. The ingredients may comprise additives to be added to a base polymer. The recipe may be generated by training (110) a machine learnable model on compound data (20) of existing (historical) compounds to predict values of compound material properties from an input recipe, providing (120) candidate recipes, selecting 1 (40) a best recipe based on a scoring function, outputting (150) the selected recipe, e.g., via a display, to enable a sample of the compound to be manufactured (200) and measured (210), receiving (160) measurement data of the sample and determining a deviation to a target specification, and determining (170) if the recipe is acceptable. If the recipe is not acceptable, the machine learned model may be retrained or updated based on the measurement data and the recipe of the sample and the above-identified steps may be repeated until a recipe meets the target specification.

### FIELD OF THE INVENTION

The invention relates to a computer-implemented method of, and processor system for, generating a recipe for a thermoplastic compound, wherein the thermoplastic compound is a compound comprising a base polymer, wherein the recipe defines a set of ingredients and a relative contribution of the ingredients for manufacturing the compound. The invention further relates to a computer-readable medium comprising a computer program for performing the computer-implemented method, to a method which additionally comprises generating a sample of the thermoplastic compound, to a method of manufacturing the thermoplastic compound using the recipe, and to a thermoplastic compound obtainable by the method.

### BACKGROUND OF THE INVENTION

Compounds, such as for example thermoplastic polymers mixed with other ingredients such as glass fibers, stabilizers, flame retardants, colorants, etc., may be manufactured according to a target specification, for example at the request of a customer. For example, the customer may request a compound to have a certain color, which color may be for example expressed as a CIELAB value and which CIELAB value may be measured on a target plate manufactured from the compound under a certain light source. For the compound to meet the target specification, either fully or at least to an acceptable degree, a recipe for the manufacture of the compound may be determined. Such a recipe may for example define a set of ingredients to be used in the manufacture of the compound, such as coloring agents for the manufacture of a colored polymer, and a relative contribution of the ingredients with respect to each other, such as the mass fraction of the ingredients.

It is known determine such a recipe empirically. For example, in case of a colored compound, a color specialist may look for an existing color plate in his/her (physical) database that visually is nearest to the customer's color target specification. The recipe for the existing color plate, that is, the compound from which the plate is manufactured, may be known. Empirically and using smart reasoning, the color specialist may generate a coloring agent recipe using the recipe of the best matching color plate as a starting point. A test plate may then be made with this coloring agent recipe, for example by compounding (extrusion) and injection molding and the color values may be measured under the same light source as the customer target plate. When color values are too far off, the development cycle may repeat itself until the deviation of the color values to the target specification are acceptable.

However, the target specification may in many cases not only extend to one material property, e.g., to the color, but to several material properties of the compound, for example mechanical properties, UV stability performance, heat ageing performance, viscosity, CTI (Comparative Tracking Index) value, flammability, etc., which material properties may elsewhere also be referred to as ‘compound material properties’. For example, in case of a colored compound, when the color target specification is met, several mechanical tests may be done on samples of the colored compound, and when the mechanical properties of the colored compound deviate too much from target values for the mechanical properties, the development process may repeat itself until both the color values and the mechanical properties of the compound are according to the target specification. It may frequently occur that the mechanical properties are insufficient when optimizing for color. For example, for colored compounds based on high temperature polymers, hard inorganic pigments, which may be added to a base polymer to obtain a desired color, may cause glass fibers in the compound to be broken down during compounding, which may result in a decrease in mechanical properties. Also, in general, when optimizing a recipe for a compound to meet the target specification with respect to one material property, other material properties may be affected as well by the changes in the recipe during the optimization and may therefore also need to be checked for adequacy.

The above-described development process may be time-consuming, expensive, may have a long throughput time, while on the other hand, the target specifications may not always be met. Examples of typical difficulties that may be encountered during for example a color matching process are the selection of the best existing recipe to use as a starting point and the estimation of the effect of a change in coloring agent on the color of the compound. Similar difficulties may be encountered when optimizing for other material properties, such as mechanical properties.

It may thus be desirable to be able to generate a recipe for the manufacture of a compound which may be less time-consuming and/or have a shorter throughput time.

### SUMMARY OF THE INVENTION

In accordance with a first aspect of the invention, a computer-implemented method is provided for generating a recipe for a thermoplastic compound, wherein the thermoplastic compound is a compound comprising a base polymer, wherein the recipe defines a set of ingredients and a relative contribution of the ingredients for manufacturing the compound, wherein the set of ingredients comprises one or more additives to be added to the base polymer, comprising:
obtaining compound data of compounds generated using known recipes, wherein the compound data comprises, for a respective compound, a) measurement values of one or more compound material properties and b) a recipe, of the respective compound;
training a machine learnable model on the compound data to predict values of the one or more compound material properties from an input recipe, thereby obtaining a predictive model;
generating the recipe for the compound to approximate target values for the one or more compound material properties by:
i) generating candidate recipes for the compound and using the predictive model to predict the values of the one or more compound material properties for each candidate recipe;
ii) selecting a recipe from the candidate recipes, wherein the selecting of the recipe comprises evaluating a scoring function, wherein the scoring function is configured to reward a correspondence between said predicted values of the one or more compound material properties of a candidate recipe and the target values;
iii) outputting said selected recipe to enable a sample of the compound to be generated and the one or material properties of the sample to be measured;
iv) receiving sample measurement values of the one or material properties of the sample and comparing the sample measurement values to the target values to determine a deviation with respect to the target values; and
v) if the deviation meets an acceptability criterion, outputting (170) the selected recipe as the recipe for the compound.

In accordance with a further aspect of the invention, a transitory or non-transitory data computer-readable medium is provided, wherein the computer-readable medium comprises data representing a computer program, wherein the computer program comprises instructions for causing a processor system to perform any one of the computer-implemented methods described in this specification.

In accordance with a further aspect of the invention, a processor system is provided for generating a recipe for a thermoplastic compound, wherein the thermoplastic compound is a compound comprising a base polymer, wherein the recipe defines a set of ingredients and a relative contribution of the ingredients to the recipe for manufacturing the compound, wherein the set of ingredients comprises one or more additives to be added to the base polymer, comprising:
a data storage interface configured for accessing compound data of compounds generated using known recipes, wherein the compound data comprises, for a respective compound, a) measurement values of one or more compound material properties and b) a recipe, of the respective compound;
a processing subsystem configured to:
   train a machine learnable model on the compound data to predict values of the one or more compound material properties from an input recipe, thereby obtaining a predictive model;
   generate a recipe for a compound to approximate target values for the one or more compound material properties by:
   generating candidate recipes for the compound and using the predictive model to predict the values of the one or more compound material properties for each candidate recipe;
ii) selecting a recipe from the candidate recipes, wherein the selecting of the recipe comprises evaluating a scoring function, wherein the scoring function is configured to reward a correspondence between said predicted values of the one or more compound material properties of a candidate recipe and the target values;
iii) outputting said selected recipe to enable a sample of the compound to be generated and the one or material properties of the sample to be measured;
iv) receiving sample measurement values of the one or material properties of the sample and comparing the sample measurement values to the target values to determine a deviation with respect to the target values;
v) if the deviation meets an acceptability criterion, outputting the selected recipe as the recipe for the compound.

The above measures may involve generating a recipe for the manufacture of a thermoplastic compound. The recipe may define a set of ingredients for the compound which may be used in the manufacture of the compound. Such ingredients may comprise additives to be added to a base polymer, for example coloring agents (‘colorants’) such as dyes and pigments, glass fibers, stabilizers, flame retardants, processing aids, flow improving agents, etc. In some examples, the recipe may define all ingredients of the compound including the base polymer, while in other examples, the recipe may only define the additives to be added to the base polymer or to the base polymer and other base components. An example of the latter type of recipe is a recipe which defines which coloring agents to add to a base polymer so as to obtain a colored polymer. The recipe may further define a relative contribution of the ingredients, for example as mass fractions. It is noted that recipes of the type defined in this paragraph are known per se. For avoidance of any doubt, it is further noted that a set of ingredients may comprise only a single ingredient, in that the recipe may identify one ingredient to be added and the amount of the ingredient to be added. In other words, the set may be a singleton set.

The above measures may further involve obtaining compound data of compounds which were previously generated using known recipes. Such compound data may elsewhere also be referred to as ‘historical’ compound data as it may pertain to existing compounds, i.e., compounds having been previously manufactured, for example by mass production or as individual samples. Of these existing compounds, one or more compound material properties may have been measured. With continued reference to the example given in the background section, the color value of a compound may have been measured, for example using a test plate which was manufactured of the compound. In addition, the recipe of such an existing compound may be known, for example by the compound having been internally manufactured using the recipe or such a recipe having been obtained in any other way. Such compound data may for example be retrieved from an existing database, which may be either a physical database, a digital database or a combination of both, and which database was built up over time during the process of generating recipes for compounds, for example empirically, manufacturing samples of the compounds and measuring the one or more compound material properties of the samples. In some examples, providing the compound data may comprise digitizing data which previously existed only in physical form or structuring existing digital data such that both the measured material properties and the recipe of the compound may be jointly accessed. In other examples, providing such compound data may comprise accessing existing samples of compounds, e.g., as test plates, and measuring such existing compounds anew.

The above measures may further involve providing a machine learnable model and training the machine learnable model on the compound data. Specifically, the machine learnable model may be trained on the known recipes and on the material properties of compounds manufactured using such known recipes. By way of the training, the machine learnable model may be learned to predict values of the one or more compound material properties from a recipe provided as input to the machine learnable model. For example, the machine learnable model may comprise a neural network which may receive an existing recipe in a computer-readable form as input and which may be trained using backpropagation to predict the compound material properties of the compound manufactured using this recipe. In another example, the machine learnable model may comprise a gaussian process of which the covariance function may be chosen on the basis of the compound data. As a result of such training, a machine learned model may be obtained, which may in the following also be referred to simply as a ‘trained model’ or a ‘predictive model’, with the latter term referring to the model having been trained to predict the one or more compound material properties on the basis of a recipe provided as input.

The above measures may further involve generating a recipe for a compound for which a target specification is given. The target specification may for example be given in the form of target values for the one or more compound material properties. Such target values may for example be numerical values which may represent desired material properties, for example defining a desired color value in CIELAB or a desired tensile strength in Pa. In other examples, at least some target values may represent minimum values, in that a better value may be desired, e.g., a higher tensile strength. The target value may thus represent a boundary above or below which the compound may be deemed unacceptable.

Given the predictive model and the target specification for the compound, the recipe for the compound may be generated as follows. A number of candidate recipes may be generated for the compound. As elucidated elsewhere, such candidate recipes may for example be generated by starting from initial recipes and changing the initial recipes so as to better match the target specification. To be able to evaluate whether a change results in an improvement or not, the predictive model may be used to, given a recipe (being either an initial recipe or one which is changed), predict the values of the one or more compound material properties of the recipe. In addition, a scoring function may be provided which may quantify a suitability of this recipe for meeting the target specification. For the purpose, the scoring function may reward a correspondence between the predicted values of the one or more compound material properties of a recipe and the target values. For example, if a target material property includes the color value, this target color value may be compared to the predicted color value of the recipe so as to determine the suitability of this recipe. Namely, a large deviation to the target material property may indicate that the recipes may be less suitable or unsuitable, while a small or no deviation to the target material property may indicate that the recipe is (more) suitable. In some examples, the candidate recipes may be generated by using the scoring function, for example by improving upon randomly generated initial recipes, while in other examples, the candidate recipes may be generated in a different manner, for example by a user specifying the candidate recipes manually.

From the set of candidate recipes, a recipe may then be selected using scoring function, which candidate recipe may represent a ‘best’ recipe in accordance with the scoring function. To validate the recipe, the recipe may be output, for example in human readable form, to enable a sample of a compound to be made using the recipe. Such samples of compounds may be made using any known techniques, including but not limited to those described in this specification. Once the sample of the compound is made, the one or more compound material properties of the sample may be measured, for example under the same or similar conditions under which the target specification of the compound is defined, and the resulting sample measurement values may be input again into the processor system or computer-implemented method. While it may have been previously predicted what the deviation with respect to the target values is, the actual measurement may confirm or not confirm this prediction. In particular, a deviation may be determined between the actually measured values and the target values, and if the deviation is acceptable, the recipe may be selected as a recipe for the compound. Such acceptability may for example be assessed by a computer-readable acceptability criterion which may for example define a tolerance range and which may be evaluated by the processor system and method. In other examples, the acceptability criterion may be a criterion of the user, with the user either approving or disapproving the recipe based on the deviation to the target values.

The above measures may have the effect that a machine learnable model is provided which may be trained on historical data to be able to predict material properties of a compound based on its recipe. As in many cases a great amount of such historical data is available or may be made available by consolidating data from various existing physical and digital databases, the machine learnable model may be able to learn relations and patterns in such data to be able to predict the material properties with a sufficient degree of accuracy. The thus selected recipe may therefore represent a better starting point than one which is empirically determined, as a user may be limited in terms of his/her ability to recognize relations and patterns in data. In particular, since the data space may be exceptionally large, for example in recipes which may comprise ingredients selected from a substantial number of possible ingredients (meaning that the recipe may be a N-dimensional vector having a large dimensionality), it may be difficult for the user to select the starting recipe for further optimization. However, it is recognized that it is possible that the selected recipe is still insufficient, for example due to the aforementioned large dimensionality of the data space, which may cause the predictive model to lack accuracy in some parts of this data space. Accordingly, the user is enabled to manufacture a sample using the recipe, as the recipe may be output by the processor system and method, to measure its material properties and to feed the resulting measurement values back into the processor system and method, so as to enable the processor system and method to determine whether the compound sufficiently meets the target specification.

As the process of generating the recipe for manufacturing the compound is now at least partially automated by way of the above measures, and in particular, by providing and using a machine learned model trained on historical compound data, it may suffice for fewer samples to be manufactured, in that the first sample may already provide a better match to the target specification than previously when relying solely on the empirical selection of a starting recipe. Advantageously, the recipe for the compound may be generated in a less time-consuming manner and/or may need a shorter throughput time.

If the deviation does not meet the acceptability criterion, the predictive model may be retrained or updated using the sample measurement values and the sample recipe and steps i)-v) may be repeated using the predictive model. If the deviation of the manufactured sample is large, and in particular if this deviation is larger than originally predicted, this may indicate that the predictive model may be insufficiently accurate, in that it may insufficiently accurately capture relations in certain parts of the data space. This may for example be the case if the amount of training data is small in relation to the size of the data space, e.g., the dimensionality of the recipe, or if the training data is unevenly distributed across the data space, e.g., when the historical compound data has only few or no compounds with certain ingredients. To address this inadequacy, the predictive model may be retrained, in that the recipe and measurement values of the sample compound may be used to update the parameter(s) of the predictive model. Such retraining may not only improve the accuracy of the predictive model when used to regenerate a recipe for a next compound, but also when further optimizing the recipe for the compound. Namely, as the deviation to the target specification may be inacceptable, the recipe may be further optimized, which may comprise again performing the steps i) to v) using the retrained predictive model. Here, the previously selected recipe for the compound may be reused, e.g., when generating the candidate recipes, but may also be discarded and the candidate recipes may be generated anew using the retrained predictive model. As the predictive model may now be more accurate in the part of the data space which is likely to be relevant for the particular compound, the recipe selection process may now be more accurate. Advantageously, the accuracy of the predictive model may be improved over time, not only in between compounds but also during the recipe generation for a particular compound.

It is noted that some predictive models may be updated to provide a more accurate prediction without a need for retraining. For example, for gaussian processes and similar predictive models which use the compound data during inference and in which the compound data therefore effectively forms part of the predictive model, the sample measurement values and the sample recipe may be added to the compound data as a new data point. Such updating may achieve a same or similar effect as retraining, but without the computational effort which is typically required for retraining a predictive model. In some embodiments, the adding of data points and the retraining may be alternated, in that after every N new data points, the predictive model may be retrained on the compound data.

The following may define optional aspects of the invention, which may denote step(s) of the computer-implemented method, and which may denote the processing subsystem of the processor system being configured to perform the respective step(s).

Optionally, the predictive model comprises a neural network and the neural network is retrained. Such retraining may comprise updating parameters of the neural network, such as weights or bias values.

Optionally, the predictive model comprises a gaussian process, wherein the gaussian process uses the compound data for inference, and wherein the gaussian process is updated by adding the sample measurement values and the sample recipe to the compound data. Accordingly, the sample measurement values and the sample recipe may be added to the input data of the gaussian process so as to update the gaussian process.

Optionally, generating the candidate recipes for the compound comprises providing a set of random recipes and generating the candidate recipes based on the set of random recipes. The candidate recipes may be generated in numerous ways, for example by providing a set of initial recipes as a ‘seed’ and improving the initial recipes using the predictive model and the scoring function. Interestingly, it has been found that randomly generated recipes may represent a well-suitable starting point, which may be advantageous since such recipes may be easily generated, e.g., computationally instead of requiring effort of a user. Namely, due to their random nature, such initial recipes may cover the recipe's data space, i.e., the data space constituted by all possible combinations and amounts of ingredients, well. This may be of particular relevance as entirely different recipes (e.g., representing datapoints in entirely different parts of the data space, e.g., recipes using entirely different ingredients and/or entirely different amounts of the ingredients) may yield similar compounds. When for example using only one recipe as a starting point, for example a user-selected recipe, the optimization process may suffer from the ‘local minimum’ problem in that the optimization process may result in a recipe in the neighborhood of the starting point being selected (with the term ‘neighborhood’ referring to a neighborhood in the N-dimensional data space of the recipe) but is unlikely to arrive at a entirely different part of the data space which may also provide an adequate, and perhaps globally superior recipe for the compound (‘globally’ referring to considering a possible combinations of ingredients). As such, by starting from a set of random recipes, the problem of local optima in the dataspace resulting in the selection of a globally sub-optimal recipe may be reduced.

Optionally, generating the candidate recipes for the compound comprises using a genetic algorithm to iteratively change the set of random recipes to obtain an improved score according to the scoring function. While the random recipes may cover the recipe's data space well, it is unlikely for a randomly generated recipe to represent the optimal recipe for the compound since such randomly generated recipes may only represent a small number of datapoints in the entire data space. While this may be addressed by increasing the amount of randomly generated recipes, or in an extreme case, considering all possible recipes, the data space may be too large for this to be feasible. For example, if fifty ingredients are available, the data space may be fifty-dimensional, which makes exhaustive assessment of recipes infeasible. Rather, a randomly generated set of recipes may be used as a starting point, e.g., a seed, to be further improved by changing individual recipes. To assess whether a change results in an improvement, the scoring function may be evaluated. Changes may be made iteratively, for example by changing an initial randomly selected recipe, and again changing the resulting recipe. There may be several of such iterations. During each iteration, the recipes may be evaluated using the predictive model and the scoring function. To structure this search for an optimum recipe efficiently, a genetic algorithm may be used which may employ evolutionary principles to iteratively optimize the best-scoring recipes while discarding the worst-scoring recipes. As such, an efficient, evolutionary local search for an optimum may be performed starting from each randomly selected datapoint in the data space. This way, the recipe's data space may be better covered, in that it may be more likely to find a globally optimal solution, i.e., a recipe.

Optionally, iteratively changing the set of random recipes comprises at least one of:
randomly changing the relative contribution of the ingredients in a recipe;
mixing two or more recipes;
randomly omitting an ingredient from a recipe;
randomly adding an ingredient to a recipe;
changing a recipe in a direction which is selected based on a previous direction of change in a previous iteration of the genetic algorithm; and
using a gradient descent technique to change a recipe towards a local minimum of the scoring function.

Some of the above changes may represent ‘crossovers’ and ‘mutations’ in a genetic algorithm and may therefore allow the best-scoring recipes to be further optimized. In some embodiments, the changes to recipe(s) may be (pseudo) random, while in other embodiments, the changes may be purposefully towards a local minimum of the scoring function. For example, a gradient descent technique may be used, which may involve evaluating the scoring function for a recipe and/or for a potential change of the recipe to determine in which direction a recipe is to be changed. Another example is that a recipe may be changed in a direction which follows the direction of change in a previous iteration. For example, if an increase in the quantity of one of the ingredients has yielded an improvement according to the scoring function in a previous iteration, the quantity may be further increased in a subsequent iteration. The type of changes to the recipe(s) may also be varied or alternated in time. For example, in one or more initial iterations, recipes may be changed (pseudo) randomly, while in later iteration(s), recipes may be purposefully changed, for example using gradient descent or by continuing change in a certain direction.

Optionally, providing the random set of recipes comprises randomly selecting the relative contribution of the ingredients. Random recipes may be generated by randomly selecting the relative contribution of these ingredients.

Optionally, randomly selecting the relative contribution of the ingredients comprises randomly setting a contribution of an ingredient to a value selected from a range, wherein the range comprises zero as lower limit and a maximum relative contribution of the ingredient in the known recipes as upper limit. The contribution of an individual ingredient, e.g., its mass fraction, may thus be limited to the maximum from the known recipes. This way, it may be avoided generating recipes which are well outside of the datapoints represented by the existing recipes, and thereby may be likely to result in the predictive model being not able to accurately predict compound material properties for such recipes.

Optionally, randomly selecting the relative contribution of the ingredients comprises randomly setting a contribution of an ingredient to zero. While the number of possible ingredients may be large, a compound may in practice have relatively few ingredients, as such few ingredients may suffice to meet the target specification and/or as fewer ingredients may positively contribute to the manufacturability of a compound. Recipes with fewer ingredients may therefore constitute more realistic starting points for a recipe. By setting the contribution of an ingredient to zero, such an ingredient may be omitted from a recipe. In some examples, the contribution of the majority of all possible ingredients may be set to zero, for example of 50%, 60%, 70%, 80%, 85% or 90% of all possible ingredients. For example, while a randomly generated recipe may comprise all possible ingredients at different and sometimes insignificant relative contributions, for example all of 50 or 100 ingredients available in total, random recipes may be generated having for example 10 or fewer ingredients, thereby serving as more realistic starting points for a recipe.

Optionally, generating the candidate recipes for the compound comprises receiving a starting recipe as input and generating the candidate recipes based on the starting recipe. In some embodiments, the recipe generation by the system and method is constrained to generate a recipe as output which comprise as a minimum the ingredients and the quantity of the ingredients of the starting recipe. In such embodiments, the recipe which is generated may thus only comprise additional ingredients and/or additional quantities of the ingredients. This way, it may be determined how to yield or approximate the target specification for the thermoplastic compound by providing one or more additives.

Optionally, the scoring function is further configured to reward at least one of:
a recipe having fewer ingredients; and
a set of ingredients in a recipe having a lower relative contribution relative to a base ingredient of the compound.

The scoring function may not only quantify the match between, on the one hand, the target values and, on the other hand, the predicted values or actually measured values of a compound but may additionally embody other preferences regarding a recipe. For example, generally, recipes having fewer ingredients may be preferred, e.g., to limit the chance of unforeseen interactions between ingredients and due to fewer ingredients contributing to the manufacturability of a compound (e.g., by having to source and stock fewer ingredients, etc.). The scoring function may thus, in addition to expressing the correspondence in material properties to the target specification, favor recipes having fewer ingredients, for example by using the number of ingredients as a negative factor in the scoring function. Likewise, for some ingredients, such as additives, it may be preferred to use as few ingredients as possible in relation to a base ingredient of the compound. This desire may also be expressed in the scoring function, for example by using the amount of ingredients relative to the base ingredient as a negative factor in the scoring function.

As elucidated above, the compound may comprise a thermoplastic polymer as base component, preferably a thermoplastic polymer having a melting temperature of at least 200° C., or 150° C., or 100° C.

As elucidated above, the recipe may define additives to be added to the base polymer. Optionally, the additives include at least one coloring agent. Optionally, the at least one coloring agent includes at least one dye or at least one pigment.

Optionally, as part of the compound data, measurement values and recipes are provided of compounds comprising only one type of coloring agent. Such compounds may thus represent ‘pure’ colored compounds in that they may only be colored by one type of coloring agent, e.g., one type of pigment. Such type of compound data may be beneficial in the training of the predictive model as the measurement values of such compounds may show the effect of a single coloring agent on the color of the compound, and effectively may represent a datapoint on a boundary of all recipes in the compound data's data space.

Optionally, the one or more compound material properties comprise:
a color of the compound, preferably defined as a color value in a perceptually uniform color space such as CIELAB and/or as a reflectance spectrum;
one or more mechanical properties of the compound, preferably one or more of: an elongation of break, a tensile strength, and a tensile modulus.

Advantageously, by training the predictive model to predict a reflectance spectrum, so-called metamerism may be avoided or at least predicted, by which compounds having the same color in terms of color value may appear to have a same color under one type of light source, e.g., one having a high color rendering index, but a different color under another type of light source, e.g., one having a low color rendering index. By predicting the reflectance curve, the appearance of the compound may be assessed for such different types of illumination, e.g., computationally without having to perform physical experiments. Advantageously, if the target specification provides a target reflectance curve, the recipe may be generated so that the compound approximates the target reflectance curve.

Optionally, the machine learnable model comprises a neural network or a gaussian process. For example, in some embodiments, the machine learnable model may be comprised of one model which may predict several material properties. In other embodiments, the machine learnable model may be comprised of several individual sub-models which may each predict a separate material property or a subset of material properties, with the sub-models together representing the machine learnable model.

Optionally, the machine learnable model is configured to provide an uncertainty quantification for the selected recipe, wherein the uncertainty quantification is output together with the selected recipe. Such an uncertainty quantification may allow a user to obtain feedback on the certainty with which the thermoplastic compound is predicted to meet the target specification. This way, the user may obtain feedback on the probability that a compound manufactured according to the selected recipe will meet the target specification. In addition, if the uncertainty is very high, this may indicate that more training data may be needed for the predictive model to improve its prediction in this part of the solution space.

Optionally, a user interface subsystem may be provided for enabling a user to interact with the processor system, the selected recipe is output via the user interface subsystem, and the sample measurement values are received via the user interface subsystem.

In a further aspect of the invention, a method of generating a recipe for a compound is provided, which method comprises executing any one of the computer-implemented methods described in this specification on a processor system, and further comprises:
the generating of the sample of the compound; and
the measuring of the one or material properties of the sample to obtain the sample measurement values of the one or material properties of the sample.

The above method may thus involve, in addition to performing any one of the computer-implemented methods described in this specification using a processor system, performing the physical steps of generating a sample of the compound and measuring the one or more material properties.

Optionally, generating the sample of the compound comprises using at least one of: a combination of extrusion and injection molding, and injection molding without extrusion.

Optionally, generating the recipe comprises at least two iterations of performing steps i) to v), wherein:
in a first iteration, the generating of the sample comprises using injection molding without extrusion, and
in a second or later iteration, the generating of the sample comprises using a combination of extrusion and injection molding.

Advantageously, a so-called ‘short feedback loop’ (or also simply ‘short loop’) may be initially used where the sample is generated using injection molding but without extrusion. By omitting the extrusion step, the sample may be generated more efficiently, e.g., requiring less time and effort (hence the adjective ‘short’). Nevertheless, the sample generated by injection molding may suffice to initially verify whether the selected recipe at least reasonably approximates the target specification. After this initial iteration, or after a number of such initial iterations, the recipe may approach the target specification, and a more accurate assessment of the recipe may be preferred. As such, the sample may then be generated using the combination of extrusion and injection molding (‘long loop’), which may better resemble an actual (mass-produced) product and may thus yield more accurate measurements, at the expense of requiring more time and effort. Advantageously, the long loop may be executed only when more accuracy is required, i.e., when approaching the target specification, while otherwise the short loop may be executed to save time and effort.

Optionally, the base polymer is a virgin polymer, a recycled polymer, a blended polymer, or a scrap polymer, a colored polymer, or a mixture of the aforementioned types of polymers. The recipe may thus define additives to be added to either a virgin polymer, a recycled polymer, a blended polymer, or a scrap polymer, or a mixture thereof, so as to meet the target specification, e.g., in terms of color and/or mechanical properties. In some embodiments, the compound may comprise more than one base polymer, in which case each base polymer may be a same or different one of the aforementioned types of polymers.

Optionally, the base polymer is a colored polymer. For example, the base polymer may be a recycled polymer having a particular color. The method and system described in this specification may be used to generate a recipe which obtains a differently colored thermoplastic compound using the colored base polymer as starting point. In such embodiments, the recipe may define coloring agent(s) to be added to obtain the desired color, and optionally to obtain desired other properties, such as mechanical properties.

Optionally, the one or more compound material properties define a target value for a color, and wherein the base polymer is selected to have a color which is nearest to the target value. This way, a starting point in terms of color may be selected which is nearest to the target value. For example, if the target value is defined as a point a CIELAB color space, a base polymer may be selected, e.g., from a database of known base polymers, which has a CIELAB color which is nearest to that point. Similarly, if the target value is defined as a reflectance spectrum, a base polymer may be selected which has a reflectance spectrum which is nearest to the target reflectance spectrum. It will be appreciated that ‘nearest’ may be quantified using any suitable metric, for example as a Euclidean distance in the 3D CIELAB color space or using a spectral metric.

Optionally, training the machine learnable model on the compound data comprises:
processing the compound data by:
selecting pairs of compounds from the compound data, each pair comprising a first compound manufactured using a first recipe and a second compound manufactured using a second recipe;
determining a third recipe for manufacturing the second compound using the first compound as base polymer, wherein the third recipe defines one or more additives to be added to the first compound to approximate values of one or more compound material properties of the second compound, wherein the third recipe is determined based on a difference between the first recipe and the second recipe;
training the machine learnable model to predict the one or more compound material properties of the second compound based on one or more compound material properties of the first compound and the third recipe as input, thereby obtaining as the predictive model a model to predict the values of the one or more compound material properties of the compound to be manufactured from a combination of a) the input recipe and b) the values of the one or more compound material properties of the base polymer.

In a further aspect of the invention, a computer-implemented method is provided of generating a recipe for a thermoplastic compound, wherein the thermoplastic compound is a compound comprising a base polymer, wherein the recipe defines a set of ingredients and a relative contribution of the ingredients for manufacturing the compound, wherein the set of ingredients comprises one or more additives to be added to the base polymer, comprising:
obtaining compound data of compounds, wherein the compounds are thermoplastic compounds, wherein the compound data comprises, for a respective compound, a) measurement values of one or more compound material properties of the respective compound, b) measurement values of one or more compound material properties of a respective base polymer, and c) a recipe defining one or more additives to be added to the respective base polymer to obtain the respective compound;
training a machine learnable model on the compound data to predict the values of the one or more compound material properties of the compound from the values of one or more compound material properties of the base polymer and an input recipe, thereby obtaining a predictive model;
generating the recipe for the compound to be manufactured to approximate target values for the one or more compound material properties by:
i) generating candidate recipes for the compound and using the predictive model to predict the values of the one or more compound material properties for each candidate recipe;
ii) selecting a recipe from the candidate recipes, wherein the selecting of the recipe comprises evaluating a scoring function, wherein the scoring function is configured to reward a correspondence between said predicted values of the one or more compound material properties of a candidate recipe and the target values; and
iii) outputting said selected recipe.

Optionally, the compound data is second compound data, further comprising:
accessing first compound data of compounds, wherein the compounds are thermoplastic compounds generated using known recipes, wherein the compound data comprises, for a respective compound, a) measurement values of one or more compound material properties and b) a recipe, of the respective compound;
generating the second compound data by:
selecting pairs of compounds from the first compound data, each pair comprising a first compound manufactured using a first recipe and a second compound manufactured using a second recipe;
determining a third recipe for manufacturing the second compound using the first compound as base polymer, wherein the third recipe defines one or more additives to be added to the first compound to approximate values of one or more compound material properties of the second compound, wherein the third recipe is determined based on a difference between the first recipe and the second recipe;
wherein the second compound data comprises, for a respective compound, a) measurement values of one or more compound material properties of the respective compound, b) measurement values of one or more compound material properties of a respective base polymer, and c) a recipe defining one or more additives to be added to the respective base polymer to obtain the respective compound.

In a further aspect of the invention, a method of manufacturing a compound using a recipe is provided, wherein the recipe is generated by any one of the methods described in this specification.

In a further aspect of the invention, a compound is provided, which compound is obtainable by a method of manufacturing a compound described in this specification.

In a further aspect of the invention, a computer-implemented method is provided for visualizing a parameter space for recipe for a compound which is to be manufactured according to a target specification, wherein the compound is a thermoplastic compound, wherein the target specification comprises target values for one or more compound material properties of the compound, comprising:
obtaining compound data of compounds, wherein the compounds are thermoplastic compounds generated using known recipes, wherein the compound data comprises, for a respective compound, a) measurement values of one or more compound material properties and b) a recipe, of the respective compound, wherein the recipe defines a set of ingredients and a relative contribution of the ingredients for manufacturing the compound;
training a machine learnable model on the compound data to predict values of the one or more compound material properties from an input recipe, thereby obtaining a predictive model;
obtaining the target values for the one or more compound material properties of the thermoplastic compound to be manufactured;
using the predictive model, visualizing a parameter space associated with the one or more compound material properties, wherein said visualizing comprises indicating one or more regions in the parameter space in which recipes yield or approximate the target values for the one or more compound material properties.

For example, the visualization may be generated by inputting recipes into the predictive model and by determining which recipes yield or approximate the target values. Another example is that the visualization may be generated using a genetic algorithm to find recipes for the compound material properties which yield or approximate the target values.

It will be appreciated by those skilled in the art that two or more of the above-mentioned embodiments, implementations, and/or optional aspects of the invention may be combined in any way deemed useful.

Modifications and variations of any processor system, any method, computer-implemented or otherwise, or any computer-readable medium, which correspond to the described modifications and variations of another one of said entities, can be carried out by a person skilled in the art on the basis of the present description.

### BRIEF DESCRIPTION OF THE DRAWINGS

These and other aspects of the invention will be apparent from and elucidated further with reference to the embodiments described by way of example in the following description and with reference to the accompanying drawings, in which

FIG. 1 shows a method for generating a recipe for a compound, which method comprises a number of computer-implemented steps and steps of generating a sample based on a selected recipe and measuring one or more material properties of the sample;

FIG. 2 shows steps for generating candidate recipes for the compound, which generating comprises providing a set of random recipes and iteratively change the set of random recipes to obtain an improved score according to a scoring function;

FIG. 3A shows a user interface element which enables a user to specify values of target material properties for the compound for which a recipe is sought;

FIG. 3B shows a user interface element showing, for a recipe which is generated using the method of FIG. 1, a prediction of the material properties;

FIG. 3C shows a user interface element which enables a user to enter measurement values of material properties of a sample generated based on the recipe;

FIG. 4 shows a processor system for generating a recipe for a compound; and

FIG. 5 shows a computer-readable medium comprising data.

It should be noted that the figures are purely diagrammatic and not drawn to scale. In the figures, elements which correspond to elements already described may have the same reference numerals.

### LIST OF REFERENCE NUMBERS AND ABBREVIATIONS

The following list of reference numbers is provided for facilitating the interpretation of the drawings and shall not be construed as limiting the claims or clauses.
20 historical compound data
40 sample compound data
100 computer-implemented method of recipe generation
110 training machine learnable model
120 generating candidate recipes
122 providing set of random recipes
124 predicting compound material properties
126 evaluating scoring function
128 select top N
130 changing mass fractions
131 mixing recipes
132 omitting ingredient
133 adding ingredient
134 keeping recipe
136 obtaining new set of recipes
138 repeating M-times
140 selecting recipe based on scoring function
150 outputting selected recipe
160 receiving measurement data and determining deviation to target
170 determining if recipe is acceptable
200 generating sample
210 measuring sample
300 processor system
310 processing subsystem
320 data storage interface
330 data storage
340 user interface subsystem
350 display
360 user input device
370 network interface
380 network
400 non-transitory computer-readable medium
410 data

### DETAILED DESCRIPTION OF EMBODIMENTS

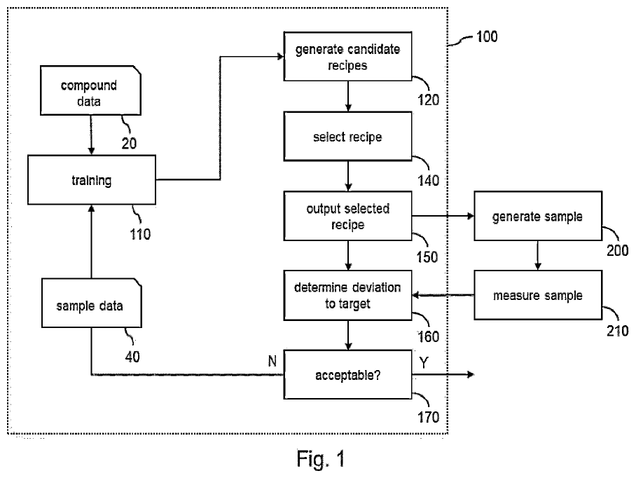

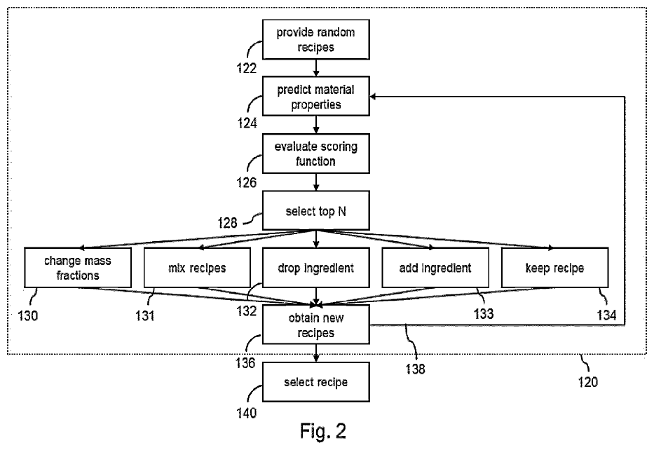

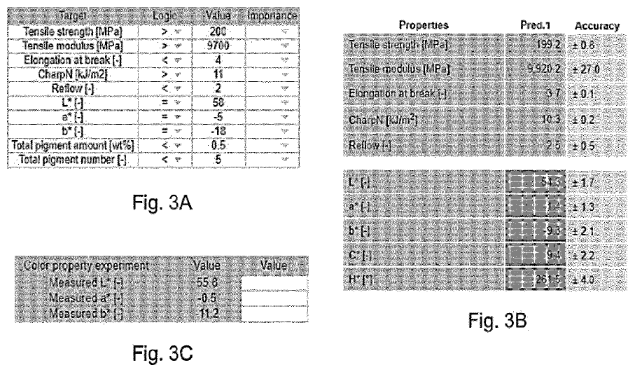

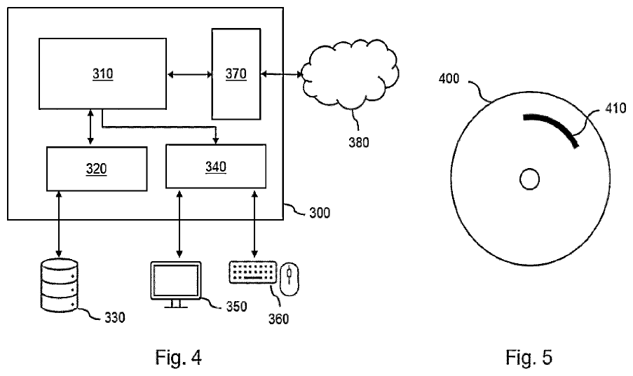

The following introduces definition used in this specification, then describes with reference to FIGS. 1-2 a method for generating a recipe for a compound, with reference to FIGS. 3A-3C a user interface for enabling a user to interact with the method or with a processor system for generating a recipe for a compound, with reference to FIG. 4 the processor system for generating the recipe for the compound and with reference to FIG. 5 a computer-readable medium storing data of embodiments described in this specification.

### Definitions

The following definitions are used in the context of the present invention.

‘Compound’: the term ‘compound’ may refer to a mixture of ingredients, such as a mixture of a base polymer with additives, with ‘base’ referring to the polymer constituting the main ingredient of the compound. The polymer may preferably be a thermoplastic polymer. The thermoplastic polymer may for example have a melting temperature of at least 200° C., or of at least 150° C., or of at least 100° C.

‘Ingredient’: the term ‘ingredient’ may refer to a component of the compound, which component may be prescribed in type and amount by the recipe. Such ingredients may include base ingredients such a base polymer, but also additives which may be added to the base ingredient, such as glass fibers, stabilizers, flame retardants, colorants (i.e., coloring agents such as pigments or dyes), processing aids, flow improving agents, etc.

‘Recipe’: a recipe may define a set of ingredients and a relative contribution of the ingredients for manufacturing the compound. The relative contribution may for example be expressed as a mass fraction or percentages. The recipe may for example be provided in computer-readable form, for example as a text file or a CSV file or in a mark-up language such as XML or as one or more entries in a database, such as a SQL-based database.

‘Manufacturing a sample of a compound’: may include any known manufacturing technique, including but not limited to injection molding, either in combination with extrusion or without extrusion, blow molding and film extrusion. Such film extrusion may for example be used to manufacture a sample of the compound by manufacturing a film of the compound for material properties which may be evaluated and measured from a film.

‘Material properties’ may include physical properties, such as mechanical properties of a compound, e.g. elongation of break, tensile strength, tensile modulus, but also non-mechanical properties such as UV stability performance, heat ageing performance, viscosity, CTI (Comparative Tracking Index) value, flammability, etc.

FIG. 1 shows a method for generating a recipe for a compound, which method comprises a number of computer-implemented steps 100, which steps may together also be referred to as a method 100, as well as a number of physical steps 200, 210. The recipe generation according to the method may be explained as follows. Firstly, compound data 20 may be obtained or generated. The compound data 20 may elsewhere be referred to as a ‘dataset’ and may comprise, for each or at least a subset of the compounds, a) measurement values of one or more compound material properties and b) a recipe, of a respective compound. A recipe and its measurement data may elsewhere also be referred to as a ‘datapoint’ in the dataset. Both the measurement values and the recipe may be provided in computer-readable form, e.g., as a text file or a CSV file or in a mark-up language such as XML or as entries in a database, such as a SQL-based database.

By way of example, in one practiced embodiment of the invention, the ingredients and CIELAB color values of more than 2000 historical recipes were obtained and structured into a database. For a number of these recipes, physical properties of the compound were measured, such as elongation of break, tensile strength, and tensile modulus. For around 1400 recipes, a full reflection curve of the compound was measured.

In some embodiments, the recipes to be included in the compound data may be selected in accordance with one or more criteria. For example, recipes may be omitted which include obsolete ingredients, e.g., ingredients which are not available anymore or which are not to be used, e.g., for regulatory reasons. Another example is that recipes with obvious errors may be omitted, for example recipes in which the sum of mass fractions is far removed from 1.0 or which have unclear/unknown ingredients. In some embodiments, as part of the compound data, measurement values and recipes of compounds comprising only one type of additive, such as a coloring agent, may be added. For example, single pigment recipes and measurements may be added to the dataset. In the aforementioned practiced embodiment, for 20 different pigments and dyes, the reflection curve and physical properties were measured at 4 different concentrations, resulting in 80 (additional) datapoints in the dataset. In some embodiments, for example those in which the compound comprises a thermoplastic polymer such as a high temperature polymer (e.g., having a melting point above 100° C. or 150° C. or 200° C.), the compound data may also specify the grade type, which grade type may be indicative of a variation in color of the compound. For example, in the aforementioned practiced embodiment, the compound data specified to which one out of 6 possible grades the thermoplastic polymer of a compound belonged to. In embodiments in which the ingredients include additives, also compounds without additives may be added to the dataset. For example, in the aforementioned practiced embodiment, natural polymers (e.g., without pigments and dyes) were added to the dataset for 6 different grades.

Having obtained the compound data 20, e.g., through (re) structuring of existing data or by anew measurements, a machine learnable model may be trained 110 on the compound data to predict values of the one or more compound material properties from an input recipe. The compound data 20 may thus constitute training data for the machine learnable model. For example, a neural network (NN) or gaussian process (GP) may be trained on the compound data 20 as training data to obtain a predictive model for predicting the material property values. In a specific example, the compound data may comprise measurement values of compound material properties such as the color of the compound and one or more mechanical properties of the compound, and the machine learnable model may be trained to predict the values of such material properties based on an input recipe. For example, in the compound data, the color may be defined as a color value in a perceptually uniform color space such as CIELAB and/or as a reflectance spectrum, while mechanical properties of the compound may be one or more of: an elongation of break, a tensile strength and a tensile modulus. The machine learnable model may thereby be learned to predict the color and the mechanical properties from a compound's recipe.

In some embodiments, the machine learnable model may also provide a confidence interval for its prediction, for example for each of the compound material properties, or for the prediction in its entirety. In some embodiments, the machine learnable model may be a single model trained to jointly predict a number of material properties. Such a model may also be referred to as a ‘combined’ model in that a combined number of material properties may be predicted, for example a combination of color values (L*, a*, b* simultaneously) or a combination of mechanical properties (tensile strength, tensile modulus, Charpy impact test score, etc.) or a combination of color values and mechanical properties. In other embodiments, the machine learnable model may be comprised of a set of models, for example individual models which may each predict an individual material property, such as a color or a mechanical property, or a set of combined models, etc. Such a machine learnable model may be referred to as a ‘composite model’. A machine learnable model may, in addition to predicting one or a small number of values of a property, also predict a large number of values, for example an entire reflection curve, which may for example enable the calculation of a CIELAB color value for any type of light source. Such a machine learnable model may also be referred to as a ‘curve model’ as it may trained to predict a reflection curve or other type of measurement curve. The machine learnable model may in general be optimized for the material properties to predict. For example, a NN model may be optimized in terms of number of layers, number of nodes and overfitting risk. A GP model may comprise a combination of two kernels and appropriate scaling of both input and output data. Such type of optimization of model architectures and parameters is known per se.

Having trained 110 the machine learnable model on the compound data 20, a recipe for a compound may be generated to approximate a target specification, that is, to approximate target values for the one or more compound material properties. For that purpose, candidate recipes may be generated 120 for the compound. Such a step of generating 120 candidate recipes may comprise using the predictive model to predict the values of the one or more compound material properties for each candidate recipe. The generating of candidate recipes may then be followed by a step of selecting 140 a recipe. Both steps may be further explained with reference to FIG. 2 which shows exemplary steps 122-138 for generating candidate recipes which are then followed by a recipe selection step 140. The steps 122-138 for generating candidate recipes may pertain to the following: the step of generating 120 such candidate recipes may be based on random recipes, in that a set of random recipes may be obtained and iteratively changed to obtain an improved score according to a scoring function. Such iterative changing may for example comprise using a genetic algorithm to iteratively change the set of random recipes to obtain an improved score according to the scoring function. When using such a genetic algorithm, but also in general, the material properties of a candidate recipe may be predicted by the predictive model, while the predicted material properties may then be evaluated by the scoring function. This quantification may enable improved variations to be created of recipes, and in particular of recipes of which the score according to the scoring function already indicates that the recipe is a promising candidate for the compound. Effectively, variants of the most promising recipes may be generated. These steps 122-138 may then be repeated until a recipe is selected 140 of which the deviation with the target specification is deemed acceptable.

With continued reference to the scoring function: such a scoring function may be provided to evaluate the suitability of predicted material properties in light of the target specification. In particular, a scoring function may be configured to reward a correspondence between the predicted values of the one or more compound material properties of a candidate recipe and the target values. Such a scoring function may take various forms. By way of example, in the aforementioned practiced embodiment, a scoring function may be provided which may be comprised of four parts, which may each be seen as a sub-score. Namely, the total score may be comprised of a color score, a physical property score, a number-of-ingredients score and a sum-of-mass-fractions-score. Mathematically, the total score may be determined as a weighted combination of these scores, e.g., as:

totalscore=a1*color_score+a2*physical_property_scores+a3*number_of_ingredients+a4*total_mass_fraction_of_pigments

Here, parameters a1, a2, a3, a4 may be optimized to, if desired, give more importance to any individual score. For example, parameters a3 and a4 may be selected as negative parameters so that the scoring function rewards recipes having fewer ingredients (i.e., the number of ingredients may represent a penalty in the calculation of the score) and recipes in which the total mass fraction of pigments relative to the mass of the base polymer is less (i.e., the total mass fraction of the pigments may represent a penalty). In this example, the scoring function may provide a higher score for a recipe which is deemed more suitable, i.e., closer to the target specification, and a lower score for a recipe which is deemed less suitable, i.e., further away from the target specification. However, this is not a limitation, in that for some types of scoring functions, a lower score may be preferred.

With continued reference to the color score: such a color score may for example expressed as a distance in the 3D CIELAB color space and may be calculate as:

$$\sqrt{{( {L}_{model} - {L}_{t a r g e t} )}^{2} + {( {a}_{model} - {a}_{t a r g e t} )}^{2} + {( {b}_{model} - {b}_{t a r g e t} )}^{2}}$$

Here, L, a, and b may represent the individual color coordinates in the CIELAB color space, while the ‘model’ color coordinates may represent those predicted by the predictive model and ‘target’ those color coordinates of the target specification. If a color distance is used as a color score as in the above example, the parameter a1 may be chosen negatively so as to reward a smaller distance and/or the penalize a higher distance.

For physical properties, such as mechanical properties, which should be above a required minimum specification, a so-called s-curve may be used with a steep increase in the score at the required minimum value. This way, a score below the minimum specification may be severely penalized while a score above the minimum specification may still be incentivized. An example of a score for a physical property (Charpy impact test score) with a target value of 10 may be the following, in which the score may be defined as a function:

```
def propscore (model,target):
  rel = (model − target)/max(target, 1)
  a = 10.0
  b = 1.0
  c = 50.0
  return a*exp(−b*exp(−c*rel))+rel
```

With continued reference to FIG. 2, in step 122, a number of random recipes may be provided. For example, such random recipes may be provided by randomly selecting the ingredients and/or the relative contribution of the ingredients for a recipe. In some embodiments, such relative contributions may be limited to a range, e.g., to define an allowable bandwidth for an ingredient's mass fraction. This range may for example run from zero as lower limit and to a maximum value. This maximum value may for example be user-defined, or may be automatically selected, for example based on a maximum relative contribution of the ingredient in the known recipes. Such a range may thus represent an allowable bandwidth for the relative contribution per ingredient. In some embodiments, a user may define the maximum value to be 0 to exclude an ingredient. In some embodiments, any of such limitation to the relative contribution may be applied to a recipe after the recipe is randomly generated. With continued reference to step 122, the relative contribution of ingredients, for example as initially randomly generated, may also be randomly set to zero. Namely, as most actual recipes have only a few ingredients, the contribution of most ingredients, e.g., after a recipe was randomly generated, may be randomly set to zero. This may allow a recipe to be always defined by the same data structure, e.g., a same vector defining the relative contribution of each possible ingredient, e.g., as a 50, 75 or 100-dimensional vector, but to limited have only few ingredients, e.g., maximum 10 or 5.

In step 124, the values of the material properties may be predicted for each randomly generated recipe using the predictive model. In step 126, the scoring function may be evaluated to determine a (numerical) score for each recipe. In steps 128-134, the recipes may be improved using a genetic algorithm. Namely, in step 128, the best recipes may be selected, for example as a top N out of M total recipes. In steps 130-133, various variations of these best recipes may be generated, for example by step 130 which may comprise randomly changing the relative contributions (e.g., the mass fraction of the ingredients in a recipe), by step 131 which may comprise mixing two or more recipes, by step 132 which may comprise randomly omitting (‘dropping’) an ingredient from a recipe, and by step 133 which may comprise randomly adding an ingredient to a recipe. By way of step 134, the best recipes themselves may also be kept, in that the new recipes obtained in step 136 may be comprised of the best recipes (e.g., the top N) and their variations generated by steps 130-133. In a specific example, 1000 recipes may initially be randomly generated. Out of the 1000 recipes, only the 200 best recipes (with the highest score) may be kept, while 800 new recipes may be created by generating variations of those 200 recipes. This may for example involve 200× randomly changing mass fractions, 200× mixing two recipes, 200× dropping a random ingredient and 200× adding a random ingredient. This process of improving recipes using a genetic algorithm may be repeated 138 a number of times, for example 50× or 100×.

With joint reference to FIGS. 1 and 2, from the candidate recipes generated by steps 122-138, a best recipe may be selected 140, for example by applying the predictive model to the most recent set of candidate recipes, e.g., as most recently generated by an iteration of steps 122-138 and selecting the best recipe according to the scoring function. In step 150, the selected recipe may then be output, e.g., in human readable form and using an output device such as a display. Based on the outputted recipe, in step 200, a sample may be generated, e.g., using manufacturing steps such as a combination of extrusion and injection molding or injection molding without extrusion. In step 210, the sample may be measured, in that the one or material properties of the sample may be measured. As a result, sample measurement values of the one or material properties of the sample may be obtained. In step 160, these measurement values may be received again by the method 100, e.g., by way of a user entering the values using a graphical user interface. Also in step 160, the sample measurement values may be compared to the target values to determine a deviation with respect to the target values. Such a deviation may be expressed in many ways, for example as a sum-of-absolute differences or a mean-squared error. In step 170, the deviation may then be evaluated against an acceptability criterion, and if the deviation meets the acceptability criterion, the selected recipe may be outputted as the recipe for the compound, e.g., using the aforementioned display or by writing the recipe as data to a data storage. An example of an acceptability criterion is a threshold, for example an absolute or relative threshold. In other examples, the deviation may be presented to a user, e.g., using the graphical user interface on the display, for the user to either accept or reject.

In some embodiments, if the deviation does not meet the acceptability criterion, the predictive model may be retrained 110 using the sample measurement values and the sample recipe, which retraining is shown in FIG. 1 as the training 110 being again performed based on sample data 40 comprising the aforementioned sample measurement values and the sample recipe. In other embodiments, and in particularly in those embodiments in which the sample data 40 is used by the predictive model during inference, instead of retraining the predictive model, the sample measurement values and the sample recipe may simply be added to the sample data 40. In both types of embodiments, the method 100 may then be repeated, for example until a recipe is obtained which is considered acceptable according to the acceptability criterion.

FIGS. 3A-3C show parts of a user interface for enabling a user to interact with the method of FIGS. 1-2 and the processor system to be described with reference to FIG. 4. Here, FIG. 3A shows a user interface element which enables a user to specify values of target material properties for the compound for which a recipe is sought. Such values may for example be entered numerically, e.g., using a keyboard, or selected graphically, e.g., using a mouse. In addition, the target values may be specified as a value to be reached, e.g., using the equal operator (‘=’), or as a minimum value, e.g., using the greater-than operator (‘>’), or as a maximum value, e.g., using the less-than operator (‘<’). Although not shown in FIG. 3A itself, the target material properties themselves may have been previously selected by the user, e.g., from a number of available target material properties, or may be predefined in the graphical user interface. In some embodiments, an importance may be selected for each target material property, which importance may be used to adjust the scoring function, for example by adjusting the aforementioned parameters a1, a2, a3, a4.

FIG. 3B shows a user interface element which shows, for a recipe which is generated using the method of FIG. 1, a prediction of the material properties. Such a user interface element may for example be used in step 150 to output a selected recipe so as to enable a sample to be generated, and/or in step 170 to verify the acceptability of a selected recipe. As can be seen in FIG. 3B, the user interface element may display the predicted values of at least some of the material properties of FIG. 3A, including their accuracy.

FIG. 3C shows a user interface element which enables a user to enter measurement values of material properties of a sample generated based on the recipe. In this example, the CIELAB values L*=55.8, a*=−0.5 and b*=−11.2 were entered. In some embodiments, several sets of values may be entered, e.g., representing several measurements of the same sample, e.g., to account for measurement variability.

Although not shown in FIG. 3A-3C, the user interface may in some embodiments present a nearest neighbor recipe of any given target specifications, with the nearest neighbor recipe being a historical recipe from the compound data of which the score according to the scoring function indicates a best approximation of the target specification, or at least part of the target specification, such as the color. Such a nearest neighbor recipe may be used to judge the improvement of a recipe generated by the genetic algorithm.

The following embodiments relate to the machine learnable model being trained to additionally use the material property (-ies) of the base polymer as input in the training and inference. In such embodiments, the training of the machine learnable model on the compound data may comprise first processing the compound data. For example, the compound data may be processed by selecting pairs of compounds from the compound data, each pair comprising a first compound manufactured using a first recipe and a second compound manufactured using a second recipe, and then determining a third recipe for manufacturing the second compound using the first compound as base polymer, wherein the third recipe defines one or more additives to be added to the first compound to approximate values of one or more compound material properties of the second compound, and wherein the third recipe is determined based on a difference between the first recipe and the second recipe. The machine learnable model may then be trained to predict the one or more compound material properties of the second compound based on one or more compound material properties of the first compound and the third recipe as input, thereby obtaining as the predictive model a model to predict the values of the one or more compound material properties of the compound to be manufactured from a combination of the input recipe and the values of the one or more compound material properties of the base polymer.

In related embodiments, a computer-implemented method may be provided for generating a recipe for a thermoplastic compound. The thermoplastic compound may be a compound comprising a base polymer, and the recipe may define a set of ingredients and a relative contribution of the ingredients for manufacturing the compound, which set of ingredients comprises one or more additives to be added to the base polymer. The method may comprise obtaining compound data of compounds, wherein the compounds are thermoplastic compounds, wherein the compound data comprises, for a respective compound, a) measurement values of one or more compound material properties of the respective compound, b) measurement values of one or more compound material properties of a respective base polymer, and c) a recipe defining one or more additives to be added to the respective base polymer to obtain the respective compound. A machine learnable model may be trained on the compound data to predict the values of the one or more compound material properties of the compound from the values of one or more compound material properties of the base polymer and an input recipe, thereby obtaining a predictive model.

As also described elsewhere, the recipe for the compound to be manufactured may be generated to approximate target values for the one or more compound material properties by generating candidate recipes for the compound and using the predictive model to predict the values of the one or more compound material properties for each candidate recipe, selecting a recipe from the candidate recipes, wherein the selecting of the recipe comprises evaluating a scoring function, wherein the scoring function is configured to reward a correspondence between said predicted values of the one or more compound material properties of a candidate recipe and the target values, and outputting said selected recipe. For example, a genetic algorithm may be used as described elsewhere.

In accordance with the above embodiments, which relate to the machine learnable model being trained to additionally use the material property (-ies) of the base polymer as input, the following is noted. The machine learnable model may be trained to predict the one or more compound material properties from a combination of an input recipe and the one or more compound material properties of the base polymer to which the input recipe pertains. Namely, the input recipe may define one or more additives to be added to a base polymer. The machine learnable model may receive the input receipt as well as the compound material property (-ies) of the base polymer as input and may be trained to predict the compound material property (-ies) of the compound to be manufactured using the input receipt. This may allow the machine learnable model to better take the starting polymer, e.g., the base polymer to which the additive(s) are added, into account, which is advantageous since there may be different starting polymers available with different characteristics. For example, virgin polymers may differ in terms of their material properties. Accordingly, when the compound to be manufactured is to have a target color, the color of the starting polymer may be taken into account when determining how to best reach the target color. It is noted that in some embodiments, the compound material property (-ies) of the base polymer may be explicitly or implicitly defined as part of the recipe. An example of the latter is that a recipe may prescribe a certain base polymer of which the material properties are known and well-defined. In such cases, it may not be needed to provide the material properties of the base polymer to the machine learnable model as input, and rather, it may suffice to provide an identifier of the base polymer to the machine learnable model.

In some embodiments, which again relate to the machine learnable model being trained to additionally use the material property (-ies) of the base polymer as input, the compound data on which the machine learnable model is trained is second compound data. In such embodiments, first compound data of compounds may be accessed, wherein the compounds are thermoplastic compounds generated using known recipes, wherein the compound data comprises, for a respective compound, a) measurement values of one or more compound material properties and b) a recipe, of the respective compound. The second compound data may then be generated by selecting pairs of compounds from the first compound data, each pair comprising a first compound manufactured using a first recipe and a second compound manufactured using a second recipe, and determining a third recipe for manufacturing the second compound using the first compound as base polymer, wherein the third recipe defines one or more additives to be added to the first compound to approximate values of one or more compound material properties of the second compound, wherein the third recipe is determined based on a difference between the first recipe and the second recipe. The second compound data may then comprise, for a respective compound, a) measurement values of one or more compound material properties of the respective compound, b) measurement values of one or more compound material properties of a respective base polymer, and c) a recipe defining one or more additives to be added to the respective base polymer to obtain the respective compound. This way, training data may be obtained for training a machine learnable model which is to be trained to predict one or more compound material properties from a combination of an input recipe and the one or more compound material properties of the base polymer to which the input recipe pertains. Obtaining such training data may be of particular relevance for the following reason:

Historical data of compounds may comprise measurements of compound material property (-ies) of compounds and recipes for manufacturing the respective compounds but may not comprise measurements of compound material property (-ies) of the base polymer used in these compounds. Rather, the recipes may simply identify the base polymer, which may historically typically be a virgin polymer. However, non-virgin polymers are of increasing relevance, e.g., for sustainability reasons, such as recycled polymers, blended polymers, or scrap polymers. These polymers may have greatly differing material properties, e.g., in terms of color, mechanical properties, etc. To be able to generate recipes using such base polymers as starting point, large amounts of new measurements may be needed, which is a complex, costly and time-intensive endeavor.

The above measures avoid needing to obtain such new measurement data by using existing historical compound data and processing the historical compound data as follows. Namely, pairs of compounds are identified the compound data, for example pairs of similar compounds in terms of material properties. The recipe of one of the compounds, which may be termed a ‘second’ compound, may contain an extra additive, or an additional quantity of an existing additive, compared to a first compound. This may mean that the second compound may be manufacturable using the first compound as starting point and by adding an extra, or an additional quantity, of an additive. In other words, the first compound may be considered as a base polymer for the second compound. The recipe defining how the second compound is to be manufactured from this base polymer may be determined by comparing the recipes of the first compound and the second compound. In a specific example, where the recipe is defined as a vector, the vector defining the recipe of the first compound may be subtracted from the vector defining the recipe of the second compound to obtain the vector for the recipe for obtaining the second compound using the first compound as base polymer. This way, it may be avoided that large amounts of new measurements have to be performed, which in turn may save cost, reduce complexity, and save time.

FIG. 4 shows a processor system 300 for generating a recipe for a compound. The processor system 300 may comprise a data storage interface 320 for reading and/or writing any type of data described in this specification from and/or to a data storage 330. The data storage 330 may for example store the compound data 20, a computer-readable version of the machine learnable model, and output data generated by the processor system 300, such as data representing a selected recipe. The data storage 330 may take various forms, such as a hard drive or an array of hard drives, a solid-state drive or an array of solid-state drives, a memory, etc. By way of example, FIG. 4 shows the data storage 330 to be an external data storage, but the data storage 330 may also be an internal component of the processor system 300. The processor system 300 may further comprise a network interface 370 to a network 380, such as a local area network (LAN) or a wide area network (WAN), such as the Internet. The network interface 370 may for example be a wired communication interface, such as an Ethernet or fiber-optic based interface, or a wireless communication interface, e.g., based on 5G, Wi-Fi, Bluetooth, ZigBee, etc. In yet other examples, the network interface 370 may be a virtual, that is, a software-defined network interface.

The processor system 300 may further comprise a processing subsystem 310 which may be configured, e.g., by hardware design or software, to perform the operations described in this specification in as far as pertaining to the generating of a recipe for a compound. In general, the processing subsystem 310 may be embodied by a single CPU, such as a x86 or ARM-based CPU, but also by a combination or system of such CPUs and/or other types of processing units, such as GPUs, NPUs, etc. In embodiments where the processor system 300 is distributed over different entities, e.g., over different servers, the processing subsystem 310 may also be distributed, e.g., over the respective CPUs, etc.

The processor system 300 may further comprise a user interface subsystem 340 which may be configured to, during operation of the processor system 300, enable a user to interact with the processor system 300, for example using a graphical user interface. In particular, the graphical user interface may enable the user to read or interpret output of the processor system 300 and to provide input, e.g., to specify target values of material properties, to enter measurement values, to approve or reject a recipe, etc. For that and other purposes, the user interface subsystem 340 may comprise a user input interface (not explicitly shown in FIG. 4) configured to receive user input data from a user input device 360 operable by the user. The user input device 360 may take various forms, including but not limited to a computer mouse, touch screen, computer keyboard, microphone, etc. FIG. 4 shows the user input device to be a computer keyboard and a computer mouse 360. In general, the user input interface may be of a type which corresponds to the type of user input device 360, i.e., it may be a thereto corresponding type of user device interface. The user interface subsystem 180 may further comprise a display output interface (not explicitly shown in FIG. 4) configured to provide display data to a display 350 to visualize output of the processor system 300. In the example of FIG. 1, the display is an external display 350. Alternatively, the display may be an internal display of the processor system 300.

In some embodiments, the processor system 300 may be a server which may operate with a client (not shown in FIG. 4) in accordance with a client-server model. In such embodiments, the graphical user interface may be presented on the client, while the information shown to the user using the graphical user interface, and input provided by the user using the graphical user interface, may be sent from and to the processor system 300. For example, the processor system 300 may be configured to establish a web-accessible graphical user interface which may be accessed by a client via the Internet or Intranet, or to interface with a webserver to establish such a graphical user interface for access by clients.

In general, each entity described in this specification may be embodied as, or in, a device or apparatus. The device or apparatus may comprise one or more (micro) processors which execute appropriate software. The processor(s) of a respective entity may be embodied by one or more of these (micro) processors. Software implementing the functionality of a respective entity may have been downloaded and/or stored in a corresponding memory or memories, e.g., in volatile memory such as RAM or in non-volatile memory such as Flash. Alternatively, the processor(s) of a respective entity may be implemented in the device or apparatus in the form of programmable logic, e.g., as a Field-Programmable Gate Array (FPGA). Any input and/or output interfaces may be implemented by respective interfaces of the device or apparatus. Each functional unit of a respective entity may be implemented in the form of a circuit or circuitry. A respective entity may also be implemented in a distributed manner, e.g., involving different devices or apparatus. An example of a device or apparatus is a computer, such as a workstation or a server.

It is noted that any of the methods described in this specification, for example in any of the claims or clauses, may be implemented on a computer as a computer-implemented method, as dedicated hardware, or as a combination of both. Instructions for the computer, e.g., executable code, may be stored on a computer-readable medium 400 as for example shown in FIG. 5, e.g., in the form of a series 410 of machine-readable physical marks and/or as a series of elements having different electrical, e.g., magnetic, or optical properties or values. The executable code may be stored in a transitory or non-transitory manner. Examples of computer-readable mediums include memory devices, optical storage devices, integrated circuits, etc. FIG. 5 shows by way of example an optical storage device 400.

The following clauses define embodiments of a computer-implemented method and processor system for generating a recipe for a compound, of a method of manufacturing the compound, and of the compound, which embodiments may be separately claimed and/or combined with other embodiments described in this specification. Clauses:

Clause 1. A computer-implemented method of generating a recipe for a compound, wherein the recipe defines a set of ingredients and a relative contribution of the ingredients for manufacturing the compound, comprising:
obtaining compound data of compounds generated using known recipes, wherein the compound data comprises, for a respective compound, a) measurement values of one or more compound material properties and b) a recipe, of the respective compound;
training a machine learnable model on the compound data to predict values of the one or more compound material properties from an input recipe, thereby obtaining a predictive model;
generating the recipe for the compound to approximate target values for the one or more compound material properties by:
i) generating candidate recipes for the compound and using the predictive model to predict the values of the one or more compound material properties for each candidate recipe;
ii) selecting a recipe from the candidate recipes, wherein the selecting of the recipe comprises evaluating a scoring function, wherein the scoring function is configured to reward a correspondence between said predicted values of the one or more compound material properties of a candidate recipe and the target values;
iii) outputting said selected recipe to enable a sample of the compound to be generated and the one or material properties of the sample to be measured;
iv) receiving sample measurement values of the one or material properties of the sample and comparing the sample measurement values to the target values to determine a deviation with respect to the target values; and
v) if the deviation meets an acceptability criterion, outputting the selected recipe as the recipe for the compound.

Clause 2. The method according to clause 1, further comprising, if the deviation does not meet the acceptability criterion, retraining the predictive model using the sample measurement values and the sample recipe and repeating steps i)-v) using the retrained predictive model.

Clause 3. The method according to clause 1 or 2, wherein generating the candidate recipes for the compound comprises providing a set of random recipes and generating the candidate recipes based on the set of random recipes.

Clause 4. The method according to clause 3, wherein generating the candidate recipes for the compound comprises using a genetic algorithm to iteratively change the set of random recipes to obtain an improved score according to the scoring function.

Clause 5. The method according to clause 4, wherein iteratively changing the set of random recipes comprises at least one of:
randomly changing the relative contribution of the ingredients in a recipe;
mixing two or more recipes;
randomly omitting an ingredient from a recipe; and
randomly adding an ingredient to a recipe.

Clause 6. The method according to any one of clauses 1 to 5, wherein providing the random set of recipes comprises randomly selecting the relative contribution of the ingredients, preferably by at least one of:
randomly setting a contribution of an ingredient to a value selected from a range, wherein the range comprises zero as lower limit and a maximum relative contribution of the ingredient in the known recipes as upper limit; and
randomly setting a contribution of an ingredient to zero.

Clause 7. The method according to any one of clauses 1 to 6, wherein the scoring function is further configured to reward at least one of:
a recipe having fewer ingredients; and
a set of ingredients in a recipe having a lower relative contribution relative to a base ingredient of the compound.

Clause 8. The method according to any one of clauses 1 to 7, wherein the one or more compound material properties comprise:
a color of the compound, preferably defined as a color value in a perceptually uniform color space such as CIELAB and/or as a reflectance spectrum;
one or more mechanical properties of the compound, preferably one or more of: an elongation of break, a tensile strength, and a tensile modulus.

Clause 9. A method of generating a recipe for a compound, comprising executing the computer-implemented method according to any one of clauses 1 to 8 on a processor system, wherein the method further comprises:
the generating of the sample of the compound; and
the measuring of the one or material properties of the sample to obtain the sample measurement values of the one or material properties of the sample.

Clause 10. The method according to clause 9, wherein generating the sample of the compound comprises using at least one of:
a combination of extrusion and injection molding; and
injection molding without extrusion.

Clause 11. The method according to clause 9 or 10, wherein generating (200) the recipe comprises at least two iterations of performing steps i) to v), wherein:
in a first iteration, the generating of the sample comprises using-injection molding without extrusion, and
in a second or later iteration, the generating of the sample comprises using a combination of extrusion and injection molding.

Clause 12. A method of manufacturing a compound using a recipe generated by the method of any one of clauses 1 to 11.

Clause 13. A compound obtainable by the method according to clause 12.

Clause 14. A transitory or non-transitory computer-readable medium comprising data representing a computer program, the computer program comprising instructions for causing a processor system to perform the method according to any one of clauses 1 to 11.

Clause 15. A processor system for generating a recipe for a compound, wherein the recipe defines a set of ingredients and a relative contribution of the ingredients to the recipe for manufacturing the compound, comprising:
a data storage interface configured for accessing compound data of compounds generated using known recipes, wherein the compound data comprises, for a respective compound, a) measurement values of one or more compound material properties and b) a recipe, of the respective compound;
a processing subsystem configured to:
   train a machine learnable model on the compound data to predict values of the one or more compound material properties from an input recipe, thereby obtaining a predictive model;
   generate a recipe for a compound to approximate target values for the one or more compound material properties by:
i) generating candidate recipes for the compound and using the predictive model to predict the values of the one or more compound material properties for each candidate recipe;
ii) selecting a recipe from the candidate recipes, wherein the selecting of the recipe comprises evaluating a scoring function, wherein the scoring function is configured to reward a correspondence between said predicted values of the one or more compound material properties of a candidate recipe and the target values;
iii outputting said selected recipe to enable a sample of the compound to be generated and the one or material properties of the sample to be measured;
iv) receiving sample measurement values of the one or material properties of the sample and comparing the sample measurement values to the target values to determine a deviation with respect to the target values; and
v) if the deviation meets an acceptability criterion, outputting the selected recipe as the recipe for the compound.

Examples, embodiments or optional features, whether indicated as non-limiting or not, are not to be understood as limiting the invention as claimed or as defined by any of the clauses.

Mathematical symbols and notations are provided for facilitating the interpretation of the invention and shall not be construed as limiting the claims or clauses.

It should be noted that the above-mentioned embodiments illustrate rather than limit the invention, and that those skilled in the art will be able to design many alternative embodiments without departing from the scope of the appended claims or clauses. In the claims or clauses, any reference signs placed between parentheses shall not be construed as limiting the claim or clause. Use of the verb “comprise” and its conjugations does not exclude the presence of elements or stages other than those stated in a claim or clause. The article “a” or “an” preceding an element does not exclude the presence of a plurality of such elements. Expressions such as “at least one of” when preceding a list or group of elements represent a selection of all or of any subset of elements from the list or group. For example, the expression, “at least one of A, B, and C” should be understood as including only A, only B, only C, both A and B, both A and C, both B and C, or all of A, B, and C. The invention may be implemented by means of hardware comprising several distinct elements, and by means of a suitably programmed computer. In the device or system claim or device or system clause enumerating several means, several of these means may be embodied by one and the same item of hardware. The mere fact that certain measures are recited in mutually different dependent claims or clauses does not indicate that a combination of these measures cannot be used to advantage.

1. A computer-implemented method (100) of generating a recipe for a thermoplastic compound, wherein the thermoplastic compound is a compound comprising a base polymer, wherein the recipe defines a set of ingredients and a relative contribution of the ingredients for manufacturing the compound, wherein the set of ingredients comprises one or more additives to be added to the base polymer, comprising:
obtaining compound data (20) of compounds, wherein the compounds are thermoplastic compounds generated using known recipes, wherein the compound data comprises, for a respective compound, a) measurement values of one or more compound material properties and b) a recipe, of the respective compound;
training (110) a machine learnable model on the compound data to predict values of the one or more compound material properties from an input recipe, thereby obtaining a predictive model;
generating the recipe for the compound to approximate target values for the one or more compound material properties by:
i) generating (120) candidate recipes for the compound and using the predictive model to predict the values of the one or more compound material properties for each candidate recipe;
ii) selecting (140) a recipe from the candidate recipes, wherein the selecting of the recipe comprises evaluating a scoring function, wherein the scoring function is configured to reward a correspondence between said predicted values of the one or more compound material properties of a candidate recipe and the target values;
iii) outputting (150) said selected recipe to enable a sample of the compound to be generated and the one or material properties of the sample to be measured;
iv) receiving (160) sample measurement values of the one or material properties of the sample and comparing the sample measurement values to the target values to determine a deviation with respect to the target values;
v) if the deviation meets an acceptability criterion, outputting (170) the selected recipe as the recipe for the compound, and if the deviation does not meet the acceptability criterion, retraining (110) or updating the predictive model using the sample measurement values and the sample recipe and repeating steps i)-v) (120-170) using the predictive model.

2. The method according to claim 1, wherein:
the predictive model comprises a neural network and the neural network is retrained; and/or
the predictive model comprises a gaussian process, wherein the gaussian process uses the compound data for inference, and wherein the gaussian process is updated by adding the sample measurement values and the sample recipe to the compound data.

3. The method (100) according to claim 1, wherein generating (120) the candidate recipes for the compound comprises providing (122) a set of random recipes and generating the candidate recipes based on the set of random recipes.

4. The method (100) according to claim 3, wherein generating (120) the candidate recipes for the compound comprises using a genetic algorithm to iteratively change (130-133) the set of random recipes to obtain an improved score according to the scoring function.

5. The method (100) according to claim 4, wherein iteratively changing the set of random recipes comprises at least one of:
randomly changing (130) the relative contribution of the ingredients in a recipe;
mixing (131) two or more recipes;
randomly omitting (132) an ingredient from a recipe;
randomly adding (133) an ingredient to a recipe;
changing a recipe in a direction which is selected based on a previous direction of change in a previous iteration of the genetic algorithm; and
using a gradient descent technique to change a recipe towards a local minimum of the scoring function.

6. The method (100) according to claim 1, wherein providing (122) the random set of recipes comprises randomly selecting the relative contribution of the ingredients, preferably by at least one of:
randomly setting a contribution of an ingredient to a value selected from a range, wherein the range comprises zero as lower limit and a maximum relative contribution of the ingredient in the known recipes as upper limit; and
randomly setting a contribution of an ingredient to zero.

7. The method (100) according to claim 1, wherein the scoring function is further configured to reward at least one of:
a recipe having fewer ingredients; and
a set of ingredients in a recipe having a lower relative contribution relative to a base ingredient of the compound.

8. The method (100) according to claim 1, wherein the one or more compound material properties comprise:
a color of the compound, preferably defined as a color value in a perceptually uniform color space such as CIELAB and/or as a reflectance spectrum;
one or more mechanical properties of the compound, preferably one or more of:
an elongation of break, a tensile strength, and a tensile modulus.

9. A method of generating a recipe for a compound, comprising executing the computer-implemented method (100) according to claim 1 on a processor system, wherein the method further comprises:
the generating (200) of the sample of the compound; and
the measuring (210) of the one or material properties of the sample to obtain the sample measurement values of the one or material properties of the sample.

10. The method according to claim 9, wherein generating (200) the sample of the compound comprises using at least one of:
a combination of extrusion and injection molding; and
injection molding without extrusion.

11. The method according to claim 9, wherein generating (200) the recipe comprises at least two iterations of performing steps i) to v) (120-170), wherein:
in a first iteration, the generating of the sample comprises using injection molding without extrusion, and
in a second or later iteration, the generating of the sample comprises using a combination of extrusion and injection molding.

12. The method according to claim 1, wherein the machine learnable model is configured to provide an uncertainty quantification for the selected recipe, wherein the method further comprises outputting the uncertainty quantification together with the selected recipe.

13. The method according to claim 1, wherein training the machine learnable model on the compound data comprises:
processing the compound data by:
selecting pairs of compounds from the compound data, each pair comprising a first compound manufactured using a first recipe and a second compound manufactured using a second recipe;
determining a third recipe for manufacturing the second compound using the first compound as base polymer, wherein the third recipe defines one or more additives to be added to the first compound to approximate values of one or more compound material properties of the second compound, wherein the third recipe is determined based on a difference between the first recipe and the second recipe;
training the machine learnable model to predict the one or more compound material properties of the second compound based on one or more compound material properties of the first compound and the third recipe as input, thereby obtaining as the predictive model a model to predict the values of the one or more compound material properties of the compound to be manufactured from a combination of a) the input recipe and b) the values of the one or more compound material properties of the base polymer.

14. The method according to claim 1, wherein the base polymer is a virgin polymer, a recycled polymer, a blended polymer, a colored polymer, or a scrap polymer.

15. The method according to claim 1, wherein the base polymer is a colored polymer.

16. A method of manufacturing a thermoplastic compound using a recipe generated by the method of claim 1.

17. A thermoplastic compound obtainable by the method according to claim 16.

18. A transitory or non-transitory computer-readable medium (400) comprising data (410) representing a computer program, the computer program comprising instructions for causing a processor system to perform the method according to claim 1.

19. A processor system (300) for generating a recipe for a thermoplastic compound, wherein the thermoplastic compound is a compound comprising a base polymer, wherein the recipe defines a set of ingredients and a relative contribution of the ingredients to the recipe for manufacturing the compound, wherein the set of ingredients comprises one or more additives to be added to the base polymer, comprising:
a data storage interface (320) configured for accessing compound data (20) of compounds, wherein the compounds are thermoplastic compounds generated using known recipes, wherein the compound data comprises, for a respective compound, a) measurement values of one or more compound material properties and b) a recipe, of the respective compound;
a processing subsystem (310) configured to:
train a machine learnable model on the compound data to predict values of the one or more compound material properties from an input recipe, thereby obtaining a predictive model;
generate a recipe for a compound to approximate target values for the one or more compound material properties by:
i) generating candidate recipes for the compound and using the predictive model to predict the values of the one or more compound material properties for each candidate recipe;
ii) selecting a recipe from the candidate recipes, wherein the selecting of the recipe comprises evaluating a scoring function, wherein the scoring function is configured to reward a correspondence between said predicted values of the one or more compound material properties of a candidate recipe and the target values;
iii) outputting said selected recipe to enable a sample of the compound to be generated and the one or material properties of the sample to be measured;
iv) receiving sample measurement values of the one or material properties of the sample and comparing the sample measurement values to the target values to determine a deviation with respect to the target values; and
if the deviation meets an acceptability criterion, outputting the selected recipe as the recipe for the compound, and if the deviation does not meet the acceptability criterion, updating the predictive model using the sample measurement values and the sample recipe and repeating steps i)-v) using the updated predictive model.

20. A computer-implemented method (100) of generating a recipe for a thermoplastic compound, wherein the thermoplastic compound is a compound comprising a base polymer, wherein the recipe defines a set of ingredients and a relative contribution of the ingredients for manufacturing the compound, wherein the set of ingredients comprises one or more additives to be added to the base polymer, comprising:
obtaining compound data (20) of compounds, wherein the compounds are thermoplastic compounds, wherein the compound data comprises, for a respective compound, a) measurement values of one or more compound material properties of the respective compound, b) measurement values of one or more compound material properties of a respective base polymer, and c) a recipe defining one or more additives to be added to the respective base polymer to obtain the respective compound;
training (110) a machine learnable model on the compound data to predict the values of the one or more compound material properties of the compound from the values of one or more compound material properties of the base polymer and an input recipe, thereby obtaining a predictive model;
generating the recipe for the compound to be manufactured to approximate target values for the one or more compound material properties by:
i) generating (120) candidate recipes for the compound and using the predictive model to predict the values of the one or more compound material properties for each candidate recipe;
ii) selecting (140) a recipe from the candidate recipes, wherein the selecting of the recipe comprises evaluating a scoring function, wherein the scoring function is configured to reward a correspondence between said predicted values of the one or more compound material properties of a candidate recipe and the target values; and
iii) outputting (150) said selected recipe.

21. The method according to claim 20, wherein the compound data is second compound data, further comprising:
accessing first compound data of compounds, wherein the compounds are thermoplastic compounds generated using known recipes, wherein the compound data comprises, for a respective compound, a) measurement values of one or more compound material properties and b) a recipe, of the respective compound;
generating the second compound data by:
selecting pairs of compounds from the first compound data, each pair comprising a first compound manufactured using a first recipe and a second compound manufactured using a second recipe;
determining a third recipe for manufacturing the second compound using the first compound as base polymer, wherein the third recipe defines one or more additives to be added to the first compound to approximate values of one or more compound material properties of the second compound, wherein the third recipe is determined based on a difference between the first recipe and the second recipe.
